(19) United States
(12) Patent Application Publication
(10) Pub. No.: US 2026/0157620 A1
(43) Pub. Date: Jun. 11, 2026

(54) SYSTEMS AND METHODS FOR MULTI-MODALITY DIAGNOSIS

(71) Applicant: Santec Holdings Corporation, Komaki (JP)

(72) Inventor: Changho Chong, Los Altos, CA (US)

(73) Assignee: Santec Holdings Corporation, Komaki (JP)

(21) Appl. No.: 18/970,494

(22) Filed: Dec. 5, 2024

(51) Int. Cl. A61B 3/00; A61B 3/14; A61B 5/00; G16H 10/60; G16H 15/00

(52) U.S. Cl. CPC A61B 3/0025; A61B 3/14; A61B 5/7267; G16H 10/60; G16H 15/00

(57) ABSTRACT

A multi-modality diagnostic system includes an optical system configured to measure an eye of a patient and a controller. The optical system includes a first optical path configured to measure the eye in a first mode and a second optical path configured to measure the eye in a second mode. The controller is configured to receive patient information from a database, determine one of the first optical path or the second optical path, based on the patient information, control the optical system to measure the eye using a determined optical path, analyze a measured image of the eye by applying a machine learning (ML) model to the measured image, and generate a report based on an analyzed image.

### BACKGROUND

The following description is provided to assist the understanding of the reader. None of the information provided or references cited is admitted to be prior art.

Various optical systems may be used to generate image data related to the health conditions of a patient. A computing device may analyze the image data.

### SUMMARY

The techniques disclosed herein provide systems and methods for multi-modality diagnosis.

One aspect of the present disclosure is directed to a multi-modality diagnostic system. The multi-modality diagnostic system includes an optical system and a controller. The optical system is configured to measure an eye of a patient. The optical system includes a first optical path configured to measure the eye in a first mode, and a second optical path configured to measure the eye in a second mode. The controller is configured to receive patient information from a database, determine one of the first optical path and the second optical path, based on the patient information, control the optical system to measure the eye using a determined optical path, analyze a measured image of the eye by applying a machine learning (ML) model to the measured image, and generate a report based on an analyzed image.

In some embodiments, in receiving the patient information from the database, the controller is configured to receive identification information of the patient, and identify the patient information related to the patient based on the identification information. In some embodiments, in determining the one of the first optical path and the second optical path, the controller is configured to identify initial suspicious information from the patient information, identify a set of disorders that are probable based on the initial suspicious information, and select the one of the first optical path and the second optical path, the one associated with at least one of the set of disorders. In some embodiments, in determining the one of the first optical path and the second optical path, the controller is configured to determine or adjust a parameter or a setting for the determined one of the first optical path and the second optical path, wherein the parameter or the setting includes one of an intensity of a diagnostic beam, a scan pattern, an imaging pattern, an imaging area, an image resolution, an imaging speed, and an image processing method. In some embodiments, the determined one of the first optical path and the second optical path is the first optical path. The controller is configured to control the optical system to measure the eye using the second optical path after controlling the optical system to measure the eye using the first optical path. In some embodiments, the controller is configured to receive, in response to measuring the eye using the first optical path, a result from the optical system, and update a parameter associated with the second optical path based on the result, prior to controlling the optical system to measure the eye using the second optical path. In some embodiments, in analyzing the measured image of the eye by applying the ML model to the measured image, the controller is configured to input the measured image to the ML model, and output an analyzed image including an indication of a suspicious feature. In some embodiments, the controller is configured to receive an intermediate result in response to inputting the measured image to the ML model, receive suspicious information associated with the suspicious feature in the intermediate result, update the intermediate result based on the suspicious information, and input the updated intermediate result to the ML model. In some embodiments, the controller is configured to send a request for referral based on the analyzed image. In some embodiments, the controller is configured to receive annotation data associated with the analyzed image, and train the ML model based on the analyzed image and the annotation data.

One aspect of the present disclosure is directed to a controller for a multi-modality diagnostic system. The controller is configured to receive patient information from a database, select, based on the patient information, one of a plurality of optical paths each of which is associated with a corresponding one of a plurality of measurement modes, control the selected one of the plurality of optical paths to measure an eye of a patient, analyze a measured image of the eye by applying a machine learning (ML) model to the measured image, and generate a report based on an analyzed image.

In some embodiments, in receiving the patient information from the database, the controller is configured to receive identification information of the patient, and identify the patient information related to the patient based on the identification information. In some embodiments, in selecting the one of the plurality of optical paths, the controller is configured to identify initial suspicious information from the patient information, identify a set of disorders that are probable based on the initial suspicious information, and select the one of the plurality of optical paths, the one associated with at least one of the set of disorders. In some embodiments, in selecting the one of the plurality of optical paths, the controller is configured to determine or adjust a parameter or a setting for the selected one of the plurality of optical paths, wherein the parameter or the setting includes one of an intensity of a diagnostic beam, a scan pattern, an imaging pattern, an imaging area, an image resolution, an imaging speed, and an image processing method. In some embodiments, the selected one of the plurality of optical paths is a first optical path, and the controller is configured to measure the eye using a second optical path after measuring the eye using the first optical path. In some embodiments, the controller is configured to receive, in response to perform a first measurement using the first optical path, a result of the first measurement, and update a parameter associated with the second optical path based on the result, prior to measuring the eye using the second optical path. In some embodiments, in analyzing the measured image of the eye by applying the ML model to the measured image, the controller is configured to input the measured image to the ML model, and output an analyzed image including an indication of a suspicious feature.

One aspect of the present disclosure is directed to a method. The method includes obtaining patient information of a patient, selecting one of a plurality of paths based on the patient information, each of the plurality of optical paths configured to measure an eye of the patient, controlling the selected one of the plurality of paths to measure the eye of the patient, analyzing a measured image of the eye by applying a machine learning model to the measured image, and generating a report based on the analyzed image.

In some embodiments, obtaining the patient information includes receiving identification information of the patient, and identifying the patient information related to the patient based on the identification information. In some embodiments, selecting the one of the plurality of optical paths includes identifying initial suspicious information from the patient information, identifying a set of disorders that are probable based on the initial suspicious information, and selecting the one of the plurality of optical paths, the one associated with at least one of the set of disorders.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 depicts a block diagram of an example multi-modality diagnostic system, in accordance with various embodiments.

FIG. 2 depicts a block diagram of an example implementation of the multi-modality diagnostic system of FIG. 1, in accordance with various embodiments.

FIG. 3 depicts a flow chart of an example process for operating a multi-modality diagnostic system, in accordance with various embodiments.

FIG. 4 depicts a flow chart of an example process for operating a multi-modality diagnostic system, in accordance with various embodiments.

FIG. 5 shows an example table that can be utilized by the multi-modality diagnostic system, in accordance with various embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Eye exams are regularly performed (e.g., annually for prescribing lenses, etc.). Data collected from eye exams can be used not only for prescribing lenses and correcting vision but also for providing insights that support more comprehensive health check-ups and guidance for severe ocular diseases and disorders, such as hyper-tension and diabetes, etc. However, it is challenging for a practice to integrate these services or provide more detailed reviews. For example, a practice with a high volume of work or a busy schedule finds it difficult to offer additional services or to perform more thorough reviews, which may require expertise and capabilities in various modalities.

It should be appreciated, therefore, that a multi-modality diagnostic system, which utilizes various modalities based on collected data and analyzes data measured by the modality to diagnose the health condition of a patient, is of interest. Techniques disclosed herein include a multi-modality diagnostic system including an optical system to measure an eye of a patient and a controller. The optical system includes a first optical path configured to measure the eye in a first mode (e.g., anterior chamber imaging) and a second optical path configured to measure the eye in a second mode (e.g., retinal imaging). The controller is configured to receive patient information from a database, determine one of the first optical path or the second optical path, based on the patient information, control the optical system to measure the eye using a determined optical path, analyze a measured image of the eye by applying a machine learning (ML) model to the measured image, and generate a report based on an analyzed image.

The multi-modality diagnostic system disclosed herein can navigate and streamline the workflow of eye exams, thereby providing comprehensive care recommendations for patients. The system can include interconnected optical systems and data processing systems (e.g., physically or via a network, cloud service, etc.). For example, the multi-modality diagnostic system can include multi-modality diagnostic equipment, which can be connected to a data processing system that can aggregate patient information (e.g., patient records, a history of health conditions, etc.). The patient information can be used to automatically configure the multi-modality diagnostic equipment, selecting an appropriate diagnostic modality, measurement modes/parameters, etc. In addition, in some embodiments, the multi-modality diagnostic system can be connected to various entities (e.g., patients, medical staff, physicians, eye examiners, etc.) to communicate the patient information, diagnostic results, etc. For example, the multi-modality diagnostic system can import the measured data from the multi-modality diagnostic equipment, analyze the data, and present images in various viewing modes (e.g., OCT cross section views, 3D views, en-face (top) views, angiography mode views, fundus camera views, analysis mode views including various retinal thickness maps overlayed on other images, geometrical parameters, overlays of one mode on top of the others, segmentations on OCT, timeline views, side-by-side views, etc.) to the various entities. In some embodiments, the multi-modality diagnostic system can request referrals to physicians based on the measured/analyzed data (e.g., in response to a detection of critical indices, etc.), allowing the physicians to review the data in detail and record findings. Furthermore, the multi-modality diagnostic system can generate comprehensive health condition reports incorporating the care recommendation from physicians, which are then communicated to the patient for further action. The multi-modality diagnostic system disclosed herein can thereby enhance efficiency and accuracy by integrating various diagnostic modalities and automating equipment configuration based on patient information. This streamlines eye exams, improves communication among healthcare providers, and facilitates detailed analysis and timely referrals, ultimately supporting more comprehensive patient care and management.

Reference is now made to the figures. The figures depict various systems and methods. In alternative embodiments, one or more components or steps in the figures may be omitted or moved. It should be understood that like reference numerals can refer to like elements throughout, repetitive descriptions of which can be omitted. It should be also noted that in the drawings, the dimensions of the features are not intended to be to true scale and can be exaggerated for the sake of allowing greater understanding.

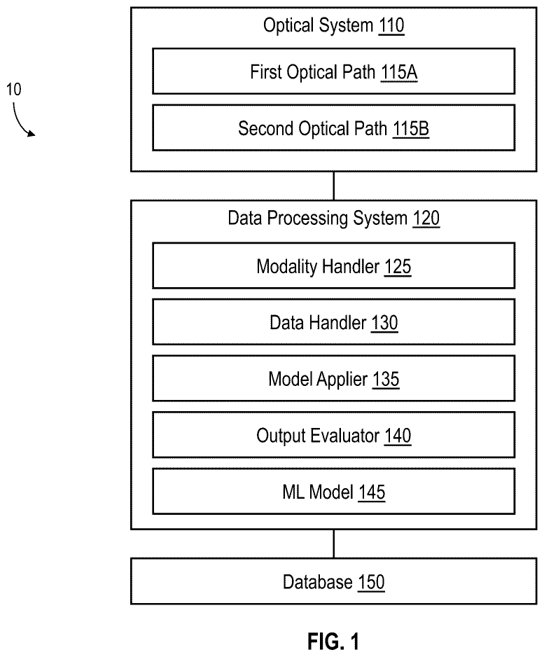

FIG. 1 depicts a block diagram of an example multi-modality diagnostic system 10, in accordance with various embodiments. The multi-modality diagnostic system 10 includes an optical system including a first optical path 115A and a second optical path 115B. The multi-modality diagnostic system 10 includes a data processing system 120 including a modality handler 125, a data handler 130, a model applier 135, an output evaluator 140, and a machine learning (ML) model 145. The multi-modality diagnostic system 10 includes a database 150. The multi-modality diagnostic system 10 shown in FIG. 1 is simplified for illustrative purposes, and thus, can be implemented as any of various other configurations while remaining within the scope of the present disclosure. In some embodiments, the multi-modality diagnostic system 10 can include more, fewer, or different components than shown in FIG. 1. For example, although depicted as including multiple optical paths, the optical system 110 can omit the first optical path 115A or the second optical path 115B as discussed in greater detail below).

The optical system 110 is a system or device including multiple optical paths (e.g., the first optical path 115A, the second optical path 115B, etc.). Each of the first optical path 115A and the second optical path 115B can be associated with a different modality, as discussed below in greater detail. The optical system 110 can output a diagnostic beam, which is to generate an image of a patient (e.g., an eye thereof), through an aperture of the multiple optical paths. For example, the optical system 110 can output a first diagnostic beam associated with the first optical path 115A and a first modality, and can output a second diagnostic beam associated with the second optical path 115B and a second modality. In some embodiments, the optical system 110 includes multiple apertures, each of which is optically connected to a corresponding one of the multiple optical paths (e.g., associated with a corresponding one of multiple modalities). In some embodiments, the optical system 110 includes a single aperture, to which each of the multiple optical paths is optically connected.

The optical system 110 can perform measurements to generate the image of the patient in various modes. In some embodiments, the first optical path 115A can be configured to measure the eye in a first mode (e.g., anterior chamber imaging or whole eye imaging as a first modality), and the second optical path 115B can be configured to measure the eye in a second mode (e.g., retinal imaging as a second modality). Although two paths are shown in FIG. 1, the optical system 110 can include any number of optical paths, which can be configured to measure the eye in different modes. In some embodiments, the first optical path 115A and/or the second optical path 115B can be associated with a modality to detect issues with vision, high glucose levels (e.g., diabetes), hypertension conditions, high cholesterol conditions, glaucoma, contact lens fitting, etc. In some embodiments, the multiple optical paths of the optical system 110 may include an optical coherence tomography (OCT) device, a phoropter, a biometry/keratometry device, an anterior OCT device, a topography device, a retinal OCT device, a fundas imaging device, an OCT angiography device, etc.

The optical system 110 can include various optical components to support the first optical path 115A and the second optical path 115B. For example, the optical system 110 can include a light source. In some embodiments, the light source may be a single swept source. The first optical path 115A and the second optical path 115B can share the single swept source. The first optical path 115A can receive a source light from the light source and then output a first diagnostic beam based on the source light. The second optical path 115B can receive a source light from the light source and then output a second diagnostic beam based on the source light. In some embodiments, the optical system 110 can include a continuous tunable laser (e.g., a tunable vertical cavity surface emitting laser (VCSEL)), which enables the optical system 110 to be reconfigurable between different modes in flexible manners, thereby accommodating the different modalities on a single platform. For example, the continuous tunable laser with VCSELs can cover a wide range of imaging depths, enabling capabilities for various types of images/modalities. In some embodiments, the optical system 110 can include various optical components configured for selective operation of the first optical path 115A and the second optical path 115B and selective output of the diagnostic beam from one of the multiple optical paths. In some embodiments, the optical system 110 can include, but not limited to, an interferometer, a detector, a switch, a mirror, etc. Example embodiments of the optical system 110 can be found in U.S. Pat. No. 9,549,671, the entire disclosure of which is incorporated by reference herein.

The data processing system 120 may be any computing device including one or more processors coupled with memory and software and configured to perform the various processes and tasks described herein. The data processing system 120 can be in communication with the optical system 110, the database 150, and other devices. In some embodiments, the data processing system 120 can be in communication with the optical system 110, the database 150, etc., physically or via a network, cloud service, etc.

The data handler 130 executing on the data processing system 120 can receive, retrieve, identify, or otherwise obtain patient information. In some embodiments, the data handler 130 can retrieve the patient information from the database 150. In some embodiments, the database 150 can be in communication with the data processing system 120 via a network. In some embodiments, the database 150 can be part (e.g., memory, etc.) of the data processing system 120. In some embodiments, the patient information may include, but not limited to, a family history (e.g., genetic, eye colors, etc.), a blood type, a medical record, a medical history (e.g., past illnesses, surgeries), current medications, allergies, lifestyle information (e.g., smoking, alcohol use, diet, BMI, exposure to sun, etc.), an immunization history, a result from a past diagnostic test/procedure, an electrocardiogram result, a urinalysis result, a treatment plan/outcome, a primary care physician, a blood test result (e.g., including glucose/cholesterol level, liver functions, etc.), health check (e.g., diabetes, glucose, blood pressure, cholesterol, visual acuity, heart/kidney disease, allergy, etc.), etc. The patient information may include image files such as Digital Imaging and Communications in Medicine (DICOM) files, etc.

In some embodiments, the data handler 130 can receive the patient information from various external sources (e.g., the patient, physicians, etc.). In some embodiments, the data handler 130 can receive the patient information through a user interface (UI) and/or a human-machine interface (HMI). For example, the data processing system 120 can be connected to or include the UI and/or HMI. A user (e.g., the patient, physicians, etc.) can provide an input and/or receive an output through the UI and/or HMI. The UI and/or HMI can include, but not limited to, a control panel (e.g., a touch screen to control the multi-modality diagnostic system 10), a display, a web/mobile application interface, a desktop software interface, etc. The UI and/or HMI can be associated with any entity (e.g., the patient, physician, etc.). In some embodiments, the data processing system 120 can identify the patient information related to a patient, in response to receiving identification information (e.g., a name, a date of birth, a gender, an age, ethnicity, etc.) of the patient. In some embodiments, the data handler 130 can receive the patient information from a networked database via a network. The data handler 130 can query the networked database and receive the patient information therefrom automatically in response to receiving the identification information. The networked database may be of any entity that can store the patient information, including but not limited to, an electronic health record (EHR), a hospital, a clinic, a primary care physician office, an urgent care center, a pharmacy, a diagnostic laboratory, an insurance company, etc. In some embodiments, the networked database may be a cloud data storage.

The modality handler 125 can configure the diagnostic modality, including selection of one of a plurality of modalities and measurement parameters. The modality handler 125 can determine one of the first optical path 115A and the second optical path 115B, based on the patient information. In some embodiments, the modality handler 125 can determine which modality (and/or which optical path) to use from a plurality of modalities (and/or a plurality of optical paths) by analyzing the patient information and identifying conditions that suggest different disorders. The modality handler 125 can identify first information in the patient information and then select a first one of the plurality of modalities (and/or a first optical path) that is associated with the first information. The modality handler 125 can identify second information in the patient information and then select a second one of the plurality of modalities (and/or a second optical path) that is associated with the second information. For example, in response to identifying that the patient is over 60 years old as the first information, the modality handler 125 may prioritize modalities that are effective in detecting cataracts. In response to identifying that the patient is over 40 years old and has a family history of glaucoma, the modality handler 125 may select an anterior chamber OCT with emphasis on angle measurement, as well as fundus and retinal OCT imaging, including the optical nerve head area for retinal thickness analysis. In response to identifying central vision loss, indicative of AMD, the modality handler 125 may opt for full retinal imaging, including fundus/OCT/OCTA. As each of the first optical path 115A and the second optical path 115B can be configured for a different modality, the modality handler 125 can select an appropriate modality based on the patient information.

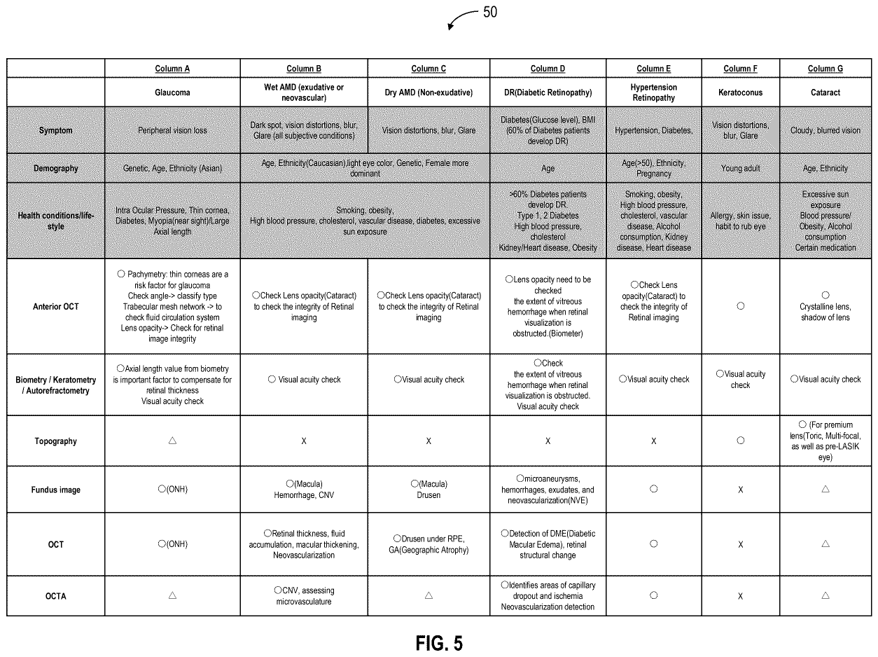

In some embodiments, the modality handler 125 can utilize a matrix of modalities associated with specific disorders and patient conditions, as discussed in greater detail below with respect to FIG. 5, to determine which modalities to use and adjust associated parameters. In some examples, the modality handler 125 can identify initial suspicion information from the patient information, and identify probable disorders based on the initial suspicion information and correlation factors. The modality handler 125 can identify factors related to different disorders. For examples, the modality handler 125 can identify the initial suspicion information based on the table shown in FIG. 5. The modality handler 125 can estimate probabilities of various disorders based on the initial suspicion information, and identify the most probable disorder. For example, the modality handler 125 can identify diabetes information from the patient information to estimate a risk of the patient to develop DR, glaucoma, etc. The modality handler 125 can identify age information from the patient information to estimate a risk of the patient to have age-related macular disease (AMD). The modality handler 125 can identify hypertension information from the patient information to estimate a risk of the patient to have hypertensive retinopathy. The modality handler 125 can identify information including age (e.g., younger demography), genetic information related to allergy, tissue condition, etc. from the patient information to estimate a risk of the patient to develop keratoconus. Discussed herein are non-limiting examples.

The modality handler 125 can select a modality based on the estimated probabilities of various disorders. The modality handler 125 can operate in various modes. In some examples, the modality handler 125 can operate to select appropriate modalities based on the estimated probabilities of various disorders. In some examples, the modality handler 125 can operate to select every modality that the optical system 110 is equipped with for wellness/prevention purpose.

The modality handler 125 can determine and/or adjust the measurement parameters and/or settings for the selected modality, based on the patient information. In some embodiments, the modality handler 125 can determine and/or adjust a set of parameters to adjust, from a plurality of parameters. For example, the parameters can include, but not limited to, an intensity of the diagnostic beam, a scan pattern, an imaging range, an imaging area, an image resolution, an imaging speed, an image processing method, etc. For example, the imaging area may be corneal area, lens, retina (ONH, Macula), vascular network (in different depth, superficial, chroidal), etc.

The modality handler 125 can control the optical system 110 to perform diagnostic measurement using the selected modality and/or measurement parameters/settings. For example, the modality handler 125 can control the optical system 110 to measure the eye of the patient using the optical path and measurement parameters selected based on the patient information. In some embodiments, the modality handler 125 can control a switch of the optical system 110 to select one of the multiple optical paths in the optical system 110. In some embodiments, the modality handler 125 can configure the selected optical path based on the measurement parameters/settings adjusted and/or determined based on the patient information. Although depicted as including the first optical path 115A and the second optical path 115B, in some embodiments, the optical system 110 can omit the second optical path 115B and perform the diagnostic measurement using a single optical path (e.g., the first optical path 115A). In this case, the modality handler 125 can configure the single optical path (e.g., the first optical path 115A) and/or associated modality based on the patient information by determining and/or adjusting the measurement parameters/settings. For example, the single optical path can be one of an OCT device, a phoropter, a biometry/keratometry device, an anterior OCT device, a topography device, a retinal OCT device, a fundas imaging device, an OCT angiography device, etc., and the modality handler 125 can determine and/or adjust the measurement parameters/settings for the single optical path.

In some examples, the modality handler 125 can control the optical system 110 to perform the diagnostic measurement in series. In some examples, the modality handler 125 can control the optical system 110 to perform a first diagnostic measurement using a first selected modality. The modality handler 125 can extract a result, a parameter, etc. from the first diagnostic measurement and link to a second modality (e.g., selected based on the patient information, and/or selected based on the result of the first diagnostic measurement, etc.). For example, the modality handler 125 can perform the first diagnostic measurement associated with biometry to obtain axial length information, and then perform the second diagnostic measurement associated with retinal imaging based on the axial length information.

The data handler 130 can receive diagnostic measurement data from the optical system 110. The diagnostic measurement data may be or include an image of the patient's eye. For example, the image includes, but not limited to, an image measured by an OCT device, a phoropter, a biometry/keratometry device, an anterior OCT device, a topography device, a retinal OCT device, a fundas imaging device, an OCT angiography device, etc. In some embodiments, the image may be DICOM image files, etc.

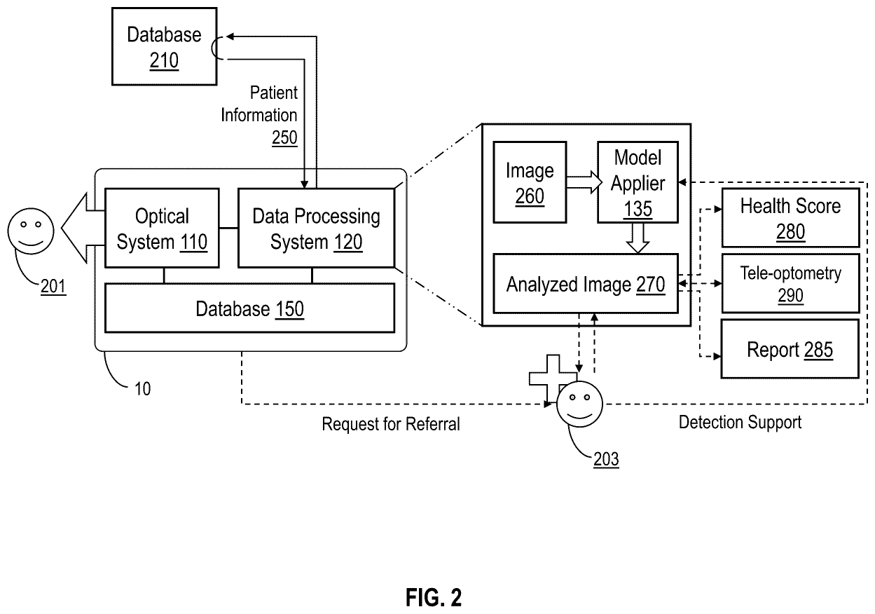

Upon receiving the diagnostic measurement data, the data processing system 120 can analyze the diagnostic measurement data based on the ML model 145. The model applier 135 executing on the data processing system 120 can apply the ML model 145 to the diagnostic measurement data. In some embodiments, the ML model 145 can be stored and/or maintained on the data processing system 120 and/or the database 150. The ML model 145 can be any type of ML algorithm or model to analyze the diagnostic measurement data measured by the optical system 110. The ML model 145 can be, for example, a deep learning artificial neural network (ANN). In some embodiments, the ML model 145 may utilize variations or hybrids of Convolutional Neural Networks (CNNs) and Vision Transformers, etc. In some embodiments, the ML model 145 can include an attention mechanism (e.g., generation of an attention map, analysis based on the attention map, etc.), as discussed in greater detail below (e.g., with respect to FIG. 2).

In some embodiments, the ML model 145 can have the diagnostic measurement data (e.g., the eye image(s)) as an input, and an indication of a suspicious feature, if any, in the diagnostic measurement data as an output, etc. In some examples, the indication of the suspicious feature may be or include a visual indication (e.g., a box surrounding a suspicious portion in the diagnostic measurement data (e.g., an image of an eye). In some examples, the indication of the suspicious feature may be or include a text, a metric, etc. For example, the indication of the suspicious feature may include probabilities of various eye disorders, characteristics of the suspicious feature (e.g., shape, dimension, etc.), etc.

The ML model 145 may have been initialized, trained, and established using a training dataset in accordance with learning techniques (e.g., supervised or semi-supervised). The training dataset can include or identify a set of examples. Each example can include a set of test measurement data and annotation data (e.g., indicating whether the test measurement data includes a suspicious feature or not), etc. The ML model 145 can use the patient information as input, in some embodiments. For example, health records can serve as labels for training the ML model 145 by correlating symptoms or indications of certain disorders with the image data. For instance, glucose levels are correlated with Diabetic Retinopathy (DR), so a patient diagnosed with diabetes can be screened for DR with the appropriate modalities. Conversely, early symptoms of DR may suggest diabetes and prompt a blood glucose test to confirm. For instance, under the supervised training method, the diagnostic measurement data from each example may be applied to the ML model 145 to generate an analysis result indicating whether a suspicious feature is found. The suspicious feature defined by the ML model 145 may be compared against the suspicious feature as defined by annotation. Based on the comparison, a degree of deviation between the feature outputted by the ML model 145 and the expected feature as defined by the annotation can be calculated. This may be used to update the ML model 145.

The output evaluator 140 executing the data processing system 105 can determine a metric for the diagnostic measurement data analyzed by the ML model 145. In some embodiments, the output evaluator 140 can, in response to the ML model 145 outputting the indication of the suspicious feature, determine the metric associated with the suspicious feature. For example, the output evaluator 140 can determine characteristics (e.g., a dimension, a shape, etc.) of the suspicious feature, etc. With the generation of the metric, the output evaluator 140 can store and maintain an association between the patient (e.g., or the diagnostic measurement data) and the metric on the database 150.

In some examples, the output evaluator 140 can generate a report based on the suspicious feature. For example, the output evaluator 140 can generate a report based on the metric (e.g., the characteristics of the suspicious features). In some embodiments, the report can include a health-risk score. The output evaluator 140 can evaluate various information. In some examples, the output evaluator 140 can generate information, including but not limited to, diagnostic information (e.g., prediction, detection of disorders, classification of disease types, severity score), prognosis information, etc. In some embodiments, the data processing system 105 can send the report to the patient. In some embodiments, the report can be in any format (e.g., a printout, a computer file, an application report, etc.).

FIG. 2 depicts a block diagram of an example implementation of the multi-modality diagnostic system 10, in accordance with various embodiments. In some embodiments, the multi-modality diagnostic system 10 can be associated with a patient 201, a physician 203, a database 210, etc. The implementation shown in FIG. 2 is simplified for illustrative purposes, and thus, can be modified with any of various other configurations while remaining within the scope of the present disclosure. In some embodiments, the implementation of the multi-modality diagnostic system 10 can be associated with more, fewer, or different components and/or entities than shown in FIG. 2.

In some embodiments, the multi-modality diagnostic system 10 can receive the identification information (e.g., a name, a date of birth, a gender, an age, ethnicity, etc.) of the patient 201. For example, the multi-modality diagnostic system 10 can receive the identification information through the HMI and/or UI from the patient 201 and/or the physician 203.

The multi-modality diagnostic system 10 can receive, retrieve, identify, or otherwise obtain patient information 250 of the patient 201. The multi-modality diagnostic system 10 can receive the patient information 250 of the patient 201 based on the identification information. In some embodiments, the multi-modality diagnostic system 10 can receive the patient information 250 of the patient 201 from the database 150 and/or the database 210. The database 210 can be of any entity that can store the patient information 250, including but not limited to, an electronic health record (EHR), a hospital, a clinic, a primary care physician office, an urgent care center, a pharmacy, a diagnostic laboratory, an insurance company, etc. For example, the multi-modality diagnostic system 10 can receive the patient information 250 of the patient 201 from the database 210 via a network. In some embodiments, the multi-modality diagnostic system 10 can query the database 210 based on the identification information of the patient 201.

The multi-modality diagnostic system 10 can configure the optical system 110 (e.g., the diagnostic modality) based on the patient information 250. In some embodiments, the multi-modality diagnostic system 10 can select one of a plurality of modalities and measurement parameters. The multi-modality diagnostic system 10 can perform the diagnostic measurement based on the selected modality and the selected measurement parameters/settings. The multi-modality diagnostic system 10 can receive diagnostic measurement data (e.g., an image 260) from the optical system 110. The image 260 may be or include an image of the patient's eye. In some embodiments, the image may be DICOM image files, etc.

The multi-modality diagnostic system 10 can analyze the image 260, based on a ML model (e.g., the ML model 145). The model applier 135 can apply the ML model to the image 260, and output an analyzed image 270. The analyzed image 270 can include an indication of a suspicious feature, if any. In some embodiments, the analyzed image 270 can further include the image 260 with an indication of a suspicious feature, if any. In some examples, the indication of the suspicious feature may be or include a visual indication (e.g., a box surrounding a suspicious portion in the image 260. In some examples, the indication of the suspicious feature may be or include a text, a metric, etc. associated with the suspicious feature identified in the image 260. For example, the indication of the suspicious feature may include probabilities of various eye disorders, characteristics of the suspicious feature (e.g., shape, dimension, etc.), etc. In some examples, in analyzing the image 260, the data processing system 120 can compare the image 260 with an image of healthy eyes.

In some embodiments, the physician 203 can support the multi-modality diagnostic system 10 for the multi-modality diagnostic system 10 to analyze the image 260 and output the analyzed image 270. For example, the physician 203 can provide annotation data (e.g., diagnostic remarks, grading, results, etc.) on the image 260, thereby improving accuracy and/or efficiency of the analysis. In some embodiments, the model applier 135 and/or the ML model can include an attention map, on which the physician 203 can annotate. The attention map may be an intermediate result that can be generated by the ML model based on the image 260. In some embodiments, the annotation of the physician 203 on the attention map can include an indication of a feature that the physician 203 finds suspicious in the image 260. In some embodiments, the intermediate result (e.g., the attention map) can include an indication of a potential disorder (e.g., visualized data, a visual indication, etc.) for attention. In some embodiments, the physician 203 can provide additional feedback on the intermediate result (e.g., the attention map). For example, the physician 203 can annotate on the intermediate result (e.g., the attention map), and/or make a visual change to the intermediate result (e.g., the attention map).

In some embodiments, the physician 203 can access the analyzed image 270 remotely. In some embodiments, the physician 203, an optometrist, an ophthalmologist, an optician, etc. can access the analyzed image 270 remotely, and provide tele-optometry service 290. For example, the physician 203 can receive the analyzed image 270 via a network. The analyzed image 270 can be displayed on an HMI/UI device (e.g., a display) of the multi-modality diagnostic system 10 or of a device of the patient 201, the physician 203, etc. In some embodiments, the multi-modality diagnostic system 10 can display the analyzed image 270 in various viewing modes (e.g., OCT cross section views, 3D views, en-face (top) views, angiography mode views, fundus camera views, analysis mode views including various retinal thickness maps overlayed on other images, geometrical parameters, overlays of one mode on top of the others, segmentations on OCT, timeline views, side-by-side views, etc.).

The multi-modality diagnostic system 10 can receive a comment regarding the analyzed image 270 from the physician 203. The comment includes, but not limited to, a diagnosis result, a care recommendation, an annotation, a label, etc. provided by the physician 203. In some embodiments, the multi-modality diagnostic system 10 can include an HMI/UI device (e.g., a display, a keyboard, a mouse, a dashboard, a web/mobile based application, etc.) that the physician 203 can use to provide the comment. In some embodiments, the multi-modality diagnostic system 10 can receive the comment from the physician 203 via a network. In response to receiving the comment from the physician 203, the multi-modality diagnostic system 10 can display information regarding the analyzed image 270 on an HMI/UI device (e.g., a display), based on the comment from the physician 203.

In some embodiments, the multi-modality diagnostic system 10 can send a request for referral to the physician 203, an optometrist, an ophthalmologist, an optician, etc. based on the analyzed image 270. The recipient of the request can send a response (e.g., to the multi-modality diagnostic system 10) to provide a recommendation for further action (e.g., an appointment, a care recommendation, etc.). In some embodiments, the multi-modality diagnostic system 10 can send a request for referral to the physician 203, an optometrist, an ophthalmologist, an optician, etc., based on a predetermined index associated with the analyzed image 270. For example, the multi-modality diagnostic system 10 can send the request for referral to the physician 203, an optometrist, an ophthalmologist, an optician, etc., in response to the analyzed image 270 including a feature that meets the predetermined index (e.g., a threshold value associated with the suspicious feature). For example, the indication of the suspicious feature can include characteristics (e.g., a size) of the suspicious feature, and the multi-modality diagnostic system 10 can send the request for referral in response to a determination that the analyzed image 270 and/or the indication of the suspicious feature including one of the characteristic that satisfy a predetermined condition (e.g., exceeding a threshold size, etc.).

In some embodiments, the multi-modality diagnostic system 10 can modify the ML model based on the comment from the physician 203 on the analyzed image 270. For example, the multi-modality diagnostic system 10 can re-train the ML model based on a set of the analyzed image 270 and the comment (e.g., annotation data on the analyzed image 270). The multi-modality diagnostic system 10 can verify a future result of the ML model based on the set of the analyzed image 270 and the comment. In some embodiments, the multi-modality diagnostic system 10 can evaluate the ML model based on the set of the analyzed image 270 and the comment. For example, the multi-modality diagnostic system 10 can evaluate a prediction accuracy of the ML model, an integrity value of the ML model, etc., based on the set of the analyzed image 270 and the comment. In some embodiments, the multi-modality diagnostic system 10 can update the ML model based on the prediction accuracy, the integrity value, etc.

The multi-modality diagnostic system 10 can generate a report 285 based on the analyzed image 270. In some embodiments, the multi-modality diagnostic system 10 can generate or update the report 285 based on the analyzed image 270 and/or the comment from the physician 203. In some embodiments, the multi-modality diagnostic system 10 can generate or update a health score 280 of the patient 201 based on the analyzed image 270 and/or the comment from the physician 203. For example, the multi-modality diagnostic system 10 can generate or update the health score indicating a health risk metric. In some embodiments, the multi-modality diagnostic system 10 can include the health score 280 in the report 285. In some embodiments, the multi-modality diagnostic system 10 can provide the report 285 and/or the health score 280 to the patient 201 (e.g., via a network).

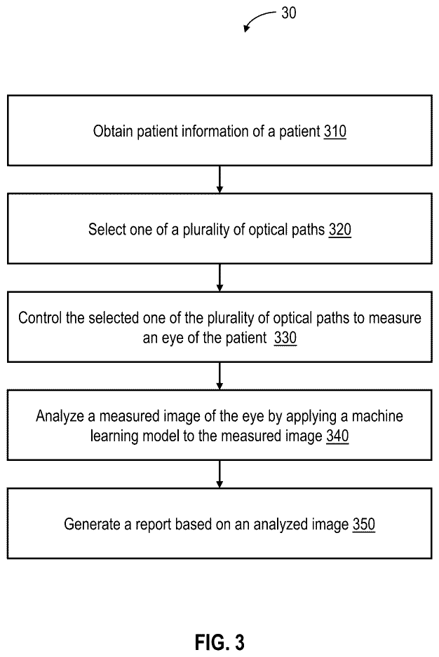

FIG. 3 depicts a flow chart of an example process 30 for operating a multi-modality diagnostic system (e.g., the multi-modality diagnostic system 10), in accordance with various embodiments. At least one of operations in the process 30 can be used to operate the multi-modality diagnostic system 10 or at least a portion thereof. It is noted that the process 30 is a non-limiting example. Accordingly, it should be understood that additional operations may be provided before, during, and/or after the process 30 of FIG. 3, and that some other operations may only be briefly described herein. In some embodiments, the process 30 can include more, fewer, or different operations than shown in FIG. 3.

In a brief overview, the process 30 begins with operation 310 of obtaining patient information of a patient. The process 30 continues to operation 320 of selecting one of a plurality of optical paths. The process 30 continues to operation 330 of controlling the selected one of the plurality of optical paths to measure an eye of the patient. The process 30 continues to operation 340 of analyzing a measured image of the eye by applying a machine learning model to the measured image. The process continues to operation 350 of generating a report based on an analyzed image.

At operation 310, patient information (e.g., the patient information 250) of a patient can be obtained. In some embodiments, a data processing system (e.g., the data handler 130) can receive, retrieve, identify, or otherwise obtain the patient information. In some embodiments, the process 30 can include, at operation 310, obtaining the patient information via a network. In some embodiments, the patient information may include, but not limited to, a family history (e.g., genetic, eye colors, etc.), a blood type, a medical record, a medical history (e.g., past illnesses, surgeries), current medications, allergies, lifestyle information (e.g., smoking, alcohol use, diet, BMI, exposure to sun, etc.), an immunization history, a result from a past diagnostic test/procedure, an electrocardiogram result, a urinalysis result, a treatment plan/outcome, a primary care physician, health check (e.g., diabetes, glucose, blood pressure, cholesterol, visual acuity, heart/kidney disease, allergy, etc.), etc. The patient information may include image files such as Digital Imaging and Communications in Medicine (DICOM), etc. In some embodiments, the process 30 can include, at operation 310, obtaining the patient information via an HMI/UI device. For example, the data processing system can receive the patient information through a user device, including but not limited to, a control panel (e.g., a touch screen), a display, a web/mobile application interface, a desktop software interface, etc. In some embodiments, at operation 310, the patient information can be obtained in response to receiving identification information (e.g., a name, a date of birth, a gender, an age, ethnicity, etc.) of a patient.

At operation 320, one of a plurality of optical paths can be selected. In some embodiments, based on the selected one of the plurality of optical paths, a diagnostic modality can be configured, including selection of one of a plurality of modalities and measurement parameters. In some embodiments, the process 30, at operation 320, can include determining one of a plurality of optical paths each of which is associated with a different modality, based on the patient information. In some embodiments, the process 30 can include, at operation 320, determining and/or adjusting the measurement parameters and/or settings for the selected modality, based on the patient information.

At operation 330, the selected one of the plurality of optical paths can be controlled to measure an eye of the patient. For example, an optical system (e.g., the optical system 110) can be controlled to perform diagnostic measurement using selected modality and/or measurement parameters. In some embodiments, the process 30 can include, at operation 330, controlling the optical system to measure an eye of the patient using the selected modality and parameters/settings. In some embodiments, the process 30 can include, at operation 330, controlling a switch of the optical system to select one of the multiple optical paths.

At operation 340, a measured image of the eye can be analyzed by applying a machine learning model to the measured image. In some embodiments, diagnostic measurement data (e.g., the image 260) can be analyzed. In some embodiments, the process 30 can include, at operation 340, analyzing the diagnostic measurement data based on an ML model (e.g., the ML model 145). The process 30 can include applying the ML model to the diagnostic measurement data and outputting analyzed data (e.g., the analyzed image 270). In some embodiments, the process 30 can include, at operation 340, outputting an indication of a suspicious feature in the diagnostic measurement data. In some embodiments, the process 30 can include, at operation 340, determining a metric associated with the suspicious feature. For example, the process 30 can include determining a dimension of the suspicious feature, etc. In some embodiments, the process 30 can include, at operation 340, recording an association between the patient and the metric. At operation 350, a report can be generated based on analysis performed at operation 340.

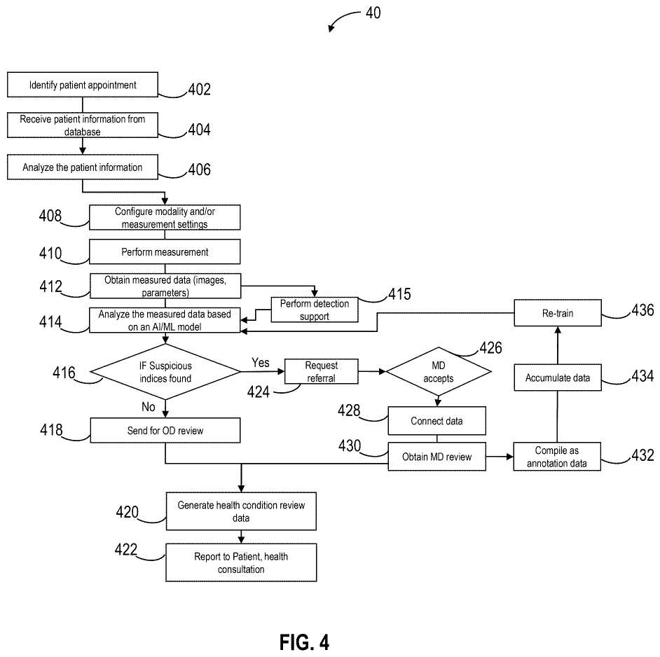

FIG. 4 depicts a flow chart of an example process 40 for operating a multi-modality diagnostic system (e.g., the multi-modality diagnostic system 10), in accordance with various embodiments. At least one of operations in the process 40 can be used to operate the multi-modality diagnostic system 10 or at least a portion thereof. It is noted that the process 40 is a non-limiting example. Accordingly, it should be understood that additional operations may be provided before, during, and/or after the process 40 of FIG. 4, and that some other operations may only be briefly described herein. In some embodiments, the process 40 can include more, fewer, or different operations than shown in FIG. 4.

In a brief overview, the process 40 begins with operation 402 of identifying a patient appointment. The process 40 continues to operation 404 of receiving patient information from a database. The process 40 continues to operation 406 of analyzing the patient information. The process 40 continues to operation 408 of configuring modality and/or measurement settings. The process 40 continues to operation 410 of performing diagnostic measurement. The process 40 continues to operation 412 of obtaining measured data (e.g., images, parameters, etc.). The process 40 continues to operation 414 of analyzing the measured data based on an AI/ML model. In some embodiments, prior to continuing to operation 414 from operation 412, the process 40 continues to operation 415 of performing detection support. The process 40 continues to operation 416, from operation 414, of determining if suspicious indices are found in the analyzed data. In response to determining that no suspicious indices are found in the analyzed data at operation 416, the process 40 continues to operation 418 of sending the analyzed data for optometry doctor (OD) review. The process 40 continues to operation 420 of generating health condition review data. process 40 continues to operation 422 of reporting health consultation to the patient. In response to determining that the suspicious indices are found in the analyzed data at operation 416, the process 40 continues to operation 424 of sending a request for referral (e.g., to a medical doctor (MD)). In response to the MD accepting the request at operation 426, the process 40 continues to operation 428 of connecting (or otherwise, providing) the analyzed data for MD review. The process 40 continues to operation 430 of obtaining the MD review, which can be incorporated into the health condition review data at operation 420. In some embodiments, the process 40 continues from operation 430 to operation 432 of completing the MD review as annotation data. The process 40 continues to operation 434 of accumulating data. The process 40 continues to operation 436 of re-training the AI/ML model.

At operation 402, a data processing system (e.g., the data processing system 120) can identify the patient appointment. The process 40 can include, at operation 402, identifying identification information of the patient including, for example, a name, a date of birth, a gender, an age, ethnicity, etc. At operation 404, the data processing system can receive patient information (e.g., the patient information 250). In some embodiments, the process 40 can include, at operation 404, receiving the patient information including, but not limited to, a family history (e.g., genetic, eye colors, etc.), a blood type, a medical record, a medical history (e.g., past illnesses, surgeries), current medications, allergies, lifestyle information (e.g., smoking, alcohol use, diet, BMI, exposure to sun, etc.), an immunization history, a result from a past diagnostic test/procedure, an electrocardiogram result, a urinalysis result, a treatment plan/outcome, a primary care physician, health check (e.g., diabetes, glucose, blood pressure, cholesterol, visual acuity, heart/kidney disease, allergy, etc.), etc. In some embodiments, the process 40 can include receiving image files (e.g., DICOM files) as the patient information.

At operation 406, the data processing system can analyze the patient information. In some embodiments, the process 40 can include, at operation 406, analyzing the patient information to determine one of a plurality of modalities (e.g., a plurality of optical paths). At operation 408, the data processing system can configure modality and/or measurement settings. In some embodiments, the process 40 can include, at operation 408, determining one of a plurality of optical paths each of which is associated with a different modality, based on the patient information. In some embodiments, the process 40 can include, at operation 320, determining and/or adjusting the measurement parameters and/or settings for the selected modality, based on the patient information. At operation 410, an optical system (e.g., the optical system 110) can be controlled to perform diagnostic measurement using selected modality and/or measurement parameters. For example, the process 40 can include, at operation 410, controlling the optical system to measure an eye of the patient using the selected modality and parameters/settings. In some embodiments, the process 40 can include, at operation 410, controlling a switch of the optical system to select one of the multiple optical paths. At operation 412, the data processing system can obtain the measured data (e.g., images, parameters, etc.) from the optical system.

At operation 414, the data processing system can analyze the measured data (e.g., the image 260) based on an AI/ML model (e.g., the ML model 145). In some embodiments, the process 40 can include, at operation 414, outputting an indication of a suspicious feature, if any, in the diagnostic measurement data. In some embodiments, the process 40 can continue to operation 415, from operation 412, of performing detection support. The process 40 can include, at operation 415, generating an intermediate result (e.g., an attention map), and receiving annotation data (e.g., diagnostic remarks, grading, results, etc.) from a physician (e.g., the physician 203). The process 40 can include incorporating the annotation data from the physician into the intermediate result. In some embodiments, the annotation can include an indication of a feature that the physician finds suspicious in the image, an indication of a potential disorder (e.g., visualized data, a visual indication, etc.) for attention, etc. At operation 414, the data processing system can analyze (e.g., using the AI/ML model) the measured data based on the intermediate result (e.g., the attention map) that includes the annotation data.

In response to a determination that the suspicious indices are not found at operation 416, the process 40 continues to operation 418 of sending the analyzed data for OD review. At operation 418, the data processing system can send the analyzed data to the OD, and receive a review result from the OD. At operation 420, the data processing system can generate a report (e.g., the report 285) including health condition review data, the OD review result, the analyzed data, etc. At operation 422, the data processing system can send the report to the patient, including health consultation, care recommendation, etc.

In response to a determination that the suspicious indices are found at operation 416, the process 40 continues to operation 424 of sending a request for referral (e.g., to an MD, the physician 203, etc.). In response to the MD accepting the request at operation 426, the process 40 continues to operation 428 of connecting the analyzed data to the MD. In some embodiments, the process 40 can include sending the analyzed data to the MD. In some embodiments, the process 40 can include authorizing the MD to access the analyzed data in the data processing system. At operation 430, the data processing system can receive, or otherwise obtain an MD review result from the MD. In some embodiments, the data processing system can incorporate the MD review result into the report (e.g., generated at operation 420) and/or the health condition review data.

In some embodiments, the process 40 can continue, from operation 430, to operation 432 of compiling the MD review result as annotation data. For example, the data processing system can compile the analyzed data and the MD review result as a data set for a training example. At operation 434, the data processing system can accumulate the compiled data. For example, the data processing system can store a plurality of data sets including the analyzed data and the MD review results on a database. At operation 436, the data processing system can re-train the AI/MD model. In some embodiments, the data processing system can update the AI/ML model that analyze the measured data at operation 414, by re-training the AI/ML model based on the accumulated data.

While discussed with respect to FIG. 1 to FIG. 4, non-limiting examples of modalities with respect to patient information are further discussed below. FIG. 5 shows an example table 50 that can be utilized by the multi-modality diagnostic system 10, in accordance with various embodiments. In some examples, the table 50 is structured as a matrix stored in the multi-modality diagnostic system 10 (e.g., the database 150) or provided thereto via a network. The multi-modality diagnostic system 10 (e.g., the modality handler 125, data handler 130, etc.) can access the table 50 to identify specific information from the patient information and determine which modality to use.

In some examples, the modality handler 125 and/or data handler 130 can identify symptom, demography, health condition/life-style, etc. as initial suspicion information from the patient information. The modality handler 125 and/or data handler 130 can determine the most probable disorder based on the initial suspicion information. Based on the a determination of the most probable disorder, the modality handler 125 can select which modality to use from a plurality of modalities (e.g., optical paths corresponding to the respective modalities), including but not limited to, anterior OCT, biometry/keratometry/autorefractometry, topography, fundus image, OCT, OCTA, etc.

Referring to Column A of the table 50, the modality handler 125 and/or data handler 130 can identify from the patient information the symptom (e.g., peripheral vision loss, etc.), demography (genetic information, age, ethnicity, etc.), health conditions/life-style (e.g., intra ocular pressure, thin cornea, diabetes, myopia (near sight)/large axial length, etc.), etc. Based on this information, the modality handler 125 and/or data handler 130 can determine glaucoma as the most probable disorder, and select an appropriate set of modalities. The modality handler 125 can select anterior OCT (e.g., to check corneal thickness, type and/or angle, integrity of retinal measurement, health of trabecular meshwork, etc.), biometry (e.g., to check axial length), keratometry, autorefractometry, fundus image (e.g., retinal image around (ONH) to check cup shape in OCT and fundus image), OCT (e.g., ONH), etc. to perform the diagnostic measurement. In some examples, the modality handler 125 can compare the measured data with data from healthy eyes. The modality handler 125 can select topography, OCTA, as needed based on the patient information. In some examples, for anterior OCT, pachymetry can be utilized to check corneal thickness, type of glaucoma (e.g., open angle, closed angle glaucoma, etc.), etc. For example, the modality handler 125 can control the optical system to check the angle and classify the type of glaucoma.

Referring to Column B of the table 50, the modality handler 125 and/or data handler 130 can identify from the patient information the symptom (e.g., dark spot, vision distortion, blur, glare, subjective conditions, etc.), demography (age, ethnicity, light eye color, genetic information, gender, etc.), health conditions/life-style (e.g., smoking, obesity, blood pressure, cholesterol, vascular disease, diabetes, excessive sun exposure, etc.), etc. Based on this information, the modality handler 125 and/or data handler 130 can determine wet AMD (e.g., exudative, neovascular, etc.) as the most probable disorder, and select an appropriate set of modalities. The modality handler 125 can select anterior OCT (e.g., to check integrity of retinal measurement), biometry, keratometry, autorefractometry, fundus image, OCT, OCTA (e.g., to check dry/wet AMD), etc. to perform the diagnostic measurement. In some examples, the data handler 130 can request for referral and/or generate a report (e.g., the report 285) indicating that a cataract surgery is needed prior to DR diagnosis based on severity of cataract.

Referring to Column C of the table 50, the modality handler 125 and/or data handler 130 can identify from the patient information the symptom (e.g., vision distortion, blur, glare, etc.), demography (age, ethnicity, light eye color, genetic information, gender, etc.), health conditions/life-style (e.g., smoking, obesity, blood pressure, cholesterol, vascular disease, diabetes, excessive sun exposure, etc.), etc. Based on this information, the modality handler 125 and/or data handler 130 can determine dry AMD (non-exudative) as the most probable disorder, and select an appropriate set of modalities. The modality handler 125 can select anterior OCT, biometry, keratometry, autorefractometry, fundus image, OCT, OCTA, etc. to perform the diagnostic measurement.

Referring to Column D of the table 50, the modality handler 125 and/or data handler 130 can identify from the patient information the symptom (e.g., diabetes, glucose level, BMI, etc.), demography (age, etc.), health conditions/life-style (e.g., blood pressure, cholesterol, kidney disease, heart disease, obesity, etc.), etc. Based on this information, the modality handler 125 and/or data handler 130 can determine diabetic retinopathy (DR) as the most probable disorder, and select an appropriate set of modalities. The modality handler 125 can select anterior OCT (e.g., to check integrity of retinal measurement), biometry, keratometry, autorefractometry, fundus image, OCT, OCTA, etc. to perform the diagnostic measurement. In some examples, the data handler 130 can request for referral and/or generate a report (e.g., the report 285) indicating that a cataract surgery is needed prior to DR diagnosis based on severity of cataract.

Referring to Column E of the table 50, the modality handler 125 and/or data handler 130 can identify from the patient information the symptom (e.g., hypertension, diabetes, etc.), demography (age, ethnicity, pregnancy, etc.), health conditions/life-style (e.g., smoking, obesity, blood pressure, cholesterol, vascular disease, alcohol consumption, kidney disease, heart disease, etc.), etc. Based on this information, the modality handler 125 and/or data handler 130 can determine hypertension retinopathy as the most probable disorder, and select an appropriate set of modalities. The modality handler 125 can select anterior OCT, biometry, keratometry, autorefractometry, fundus image, OCT, etc. to perform the diagnostic measurement. The modality handler 125 can select anterior OCT, biometry, keratometry, autorefractometry, fundus image, OCT, OCTA, etc. to perform the diagnostic measurement.

Referring to Column F of the table 50, the modality handler 125 and/or data handler 130 can identify from the patient information the symptom (e.g., vision distortions, blur, glare, etc.), demography (age, etc.), health conditions/life-style (e.g., allergy, skin issue, habit, etc.), etc. Based on this information, the modality handler 125 and/or data handler 130 can determine keratoconus as the most probable disorder, and select an appropriate set of modalities. The modality handler 125 can select anterior OCT, biometry, keratometry, autorefractometry, topography, etc. to perform the diagnostic measurement.

Referring to Column G of the table 50, the modality handler 125 and/or data handler 130 can identify from the patient information the symptom (e.g., cloudy, blurred vision, etc.), demography (age, ethnicity, etc.), health conditions/life-style (e.g., excessive sun exposure, blood pressure, obesity, alcohol consumption, certain medication, etc.), etc. Based on this information, the modality handler 125 and/or data handler 130 can determine cataract as the most probable disorder, and select an appropriate set of modalities. The modality handler 125 can select anterior OCT, biometry, keratometry, autorefractometry, topography, etc. to perform the diagnostic measurement. The modality handler 125 can select fundas image, OCT, OCTA, etc., as needed based on the patient information.

The foregoing description of illustrative embodiments has been presented for purposes of illustration and of description. It is not intended to be exhaustive or limiting with respect to the precise form disclosed, and modifications and variations are possible in light of the above teachings or can be acquired from practice of the disclosed embodiments.

While certain embodiments have been illustrated and described, it should be understood that changes and modifications can be made therein in accordance with ordinary skill in the art without departing from the technology in its broader aspects as defined in the following claims.

The embodiments, illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, compounds, compositions or systems, which can of course vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

Additional embodiments can be set forth in the following claims.

What is claimed is:

1. A multi-modality diagnostic system, comprising:
an optical system configured to measure an eye of a patient, the optical system comprising:
a first optical path configured to measure the eye in a first mode; and
a second optical path configured to measure the eye in a second mode; and
a controller configured to:
receive patient information from a database;
determine one of the first optical path and the second optical path, based on the patient information;
control the optical system to measure the eye using a determined optical path;
analyze a measured image of the eye by applying a machine learning (ML) model to the measured image; and
generate a report based on an analyzed image.

2. The multi-modality diagnostic system of claim 1, wherein in receiving the patient information from the database, the controller is configured to:
receive identification information of the patient; and
identify the patient information related to the patient based on the identification information.

3. The multi-modality diagnostic system of claim 1, wherein in determining the one of the first optical path and the second optical path, the controller is configured to:
identify initial suspicious information from the patient information;
identify a set of disorders that are probable based on the initial suspicious information; and
select the one of the first optical path and the second optical path, the one associated with at least one of the set of disorders.

4. The multi-modality diagnostic system of claim 1, wherein in determining the one of the first optical path and the second optical path, the controller is configured to:
determine or adjust a parameter or a setting for the determined one of the first optical path and the second optical path, wherein the parameter or the setting includes one of an intensity of a diagnostic beam, a scan pattern, an imaging pattern, an imaging area, an image resolution, an imaging speed, and an image processing method.

5. The multi-modality diagnostic system of claim 1,
wherein the determined one of the first optical path and the second optical path is the first optical path; and
wherein the controller is configured to:
control the optical system to measure the eye using the second optical path after controlling the optical system to measure the eye using the first optical path.

6. The multi-modality diagnostic system of claim 5, wherein the controller is configured to:
receive, in response to measuring the eye using the first optical path, a result from the optical system; and
update a parameter associated with the second optical path based on the result, prior to controlling the optical system to measure the eye using the second optical path.

7. The multi-modality diagnostic system of claim 1, wherein in analyzing the measured image of the eye by applying the ML model to the measured image, the controller is configured to:
input the measured image to the ML model; and
output an analyzed image including an indication of a suspicious feature.

8. The multi-modality diagnostic system of claim 7, wherein the controller is configured to:
receive an intermediate result in response to inputting the measured image to the ML model;
receive suspicious information associated with the suspicious feature in the intermediate result;
update the intermediate result based on the suspicious information; and
input the updated intermediate result to the ML model.

9. The multi-modality diagnostic system of claim 1, wherein the controller is configured to:
send a request for referral based on the analyzed image.

10. The multi-modality diagnostic system of claim 1, wherein the controller is configured to:
receive annotation data associated with the analyzed image; and
train the ML model based on the analyzed image and the annotation data.

11. A controller for a multi-modality diagnostic system, the controller configured to:
receive patient information from a database;
select, based on the patient information, one of a plurality of optical paths each of which is associated with a corresponding one of a plurality of measurement modes;
control the selected one of the plurality of optical paths to measure an eye of a patient;
analyze a measured image of the eye by applying a machine learning (ML) model to the measured image; and
generate a report based on an analyzed image.

12. The controller of claim 11, wherein in receiving the patient information from the database, the controller is configured to:
receive identification information of the patient; and
identify the patient information related to the patient based on the identification information.

13. The controller of claim 11, wherein in selecting the one of the plurality of optical paths, the controller is configured to:
identify initial suspicious information from the patient information;
identify a set of disorders that are probable based on the initial suspicious information; and
select the one of the plurality of optical paths, the one associated with at least one of the set of disorders.

14. The controller of claim 11, wherein in selecting the one of the plurality of optical paths, the controller is configured to:
determine or adjust a parameter or a setting for the selected one of the plurality of optical paths, wherein the parameter or the setting includes one of an intensity of a diagnostic beam, a scan pattern, an imaging pattern, an imaging area, an image resolution, an imaging speed, and an image processing method.

15. The controller of claim 11,
wherein the selected one of the plurality of optical paths is a first optical path; and
wherein the controller is configured to:
measure the eye using a second optical path after measuring the eye using the first optical path.

16. The controller of claim 15, further configured to:
receive, in response to perform a first measurement using the first optical path, a result of the first measurement; and
update a parameter associated with the second optical path based on the result, prior to measuring the eye using the second optical path.

17. The controller of claim 11, wherein in analyzing the measured image of the eye by applying the ML model to the measured image, the controller is configured to:
input the measured image to the ML model; and
output an analyzed image including an indication of a suspicious feature.

18. A method comprising:
obtaining patient information of a patient;
selecting one of a plurality of optical paths based on the patient information, each of the plurality of optical paths configured to measure an eye of the patient;
controlling the selected one of the plurality of paths to measure the eye of the patient;
analyzing a measured image of the eye by applying a machine learning model to the measured image; and
generating a report based on the analyzed image.

19. The method of claim 18, wherein obtaining the patient information includes:
receiving identification information of the patient; and
identifying the patient information related to the patient based on the identification information.

20. The method of claim 18, wherein selecting the one of the plurality of optical paths includes:
identifying initial suspicious information from the patient information;
identifying a set of disorders that are probable based on the initial suspicious information; and
selecting the one of the plurality of optical paths, the one associated with at least one of the set of disorders.
